(19) United States
(12) Patent Application Publication
(10) Pub. No.: US 2024/0252774 A1
(43) Pub. Date: Aug. 1, 2024

(54) STYLET FOR ENDOTRACHEAL INTUBATION

(71) Applicant: Diogo DE FREITAS VALEIRO GARCIA, Pinheiros, Sao Paulo (BR)

(72) Inventor: Diogo DE FREITAS VALEIRO GARCIA, Pinheiros, Sao Paulo (BR)

(21) Appl. No.: 18/560,429

(22) PCT Filed: May 10, 2022
(86) PCT No.: PCT/BR2022/050158

§ 371 Date: Nov. 13, 2023

(30) Foreign Application Priority Data
May 11, 2021 (BR) 102021009152-5

(51) Int. Cl. A61M 16/04; A61M 25/00; A61M 25/01; A61M 25/06; A61M 25/09

(52) U.S. Cl. CPC A61M 16/0488; A61M 25/0068; A61M 25/0102; A61M 2025/0681; A61M 2025/0687; A61M 25/09

(57) ABSTRACT

A device for intubating patients in a simple and quick manner, without the use of a laryngoscope. A stylet for endotracheal intubation is provided with a camera at the distal end including a rigid elongated element in a general “L” shape, having a substantially longitudinal axis through which a proximal portion for handling is configured. A distal portion forms an angle of 50° to 90° with the proximal portion, forming a knee which joins the proximal portion and distal portion. The stylet also has a semi-open channel along its entire length which leaks at the distal end and the proximal end. Said semi-open channel allows a guide wire to be attached and detached. The present invention also has an internal bidirectional working channel which is continuous along the entire length of the stylet and has an opening in the distal portion for cleaning, aspirating and introducing anesthetics.

### INVENTION FIELD

The present invention belongs to the field of medical sciences, more particularly a medical device used for endotracheal or orotracheal intubation of patients. More specifically, the invention discloses a device for intubating patients in a simple and quick manner, dispensing with the use of a laryngoscope.

### INVENTION HISTORY

Endotracheal or orotracheal intubation is a medical procedure for inserting a probe or tube into the patient's trachea through their mouth and larynx.

Patient intubation is necessary in cases such as respiratory arrest, respiratory failure, glottic edema, as well as in cases of airway obstruction, the presence of secretions or when there are gas exchange abnormalities in general, among other clinical conditions. In most surgeries using general anesthesia, it is also necessary to intubate the patient.

Intubation is usually performed with a laryngoscope to visualize mainly the vocal cords for the passage of the tube through the larynx until it reaches the patient's trachea.

Due to anatomical issues in some patients, intubation can be a more or less difficult procedure. The most challenging patients to intubate are known as difficult airway patients (DA).

Two scales are commonly used to assess the difficulty of intubating a patient: the Mallampati and Cormack classifications.

Both are graded from I to IV. In the Mallampati classification, the classification is conducted with the patient in a sitting position with the observer in front of them and looking at eye level. Thus, intubation difficulty is assessed according to the following:
Class I—soft palate, fauce, uvula and tonsillar pillars visible;
Class II—soft palate, fauce and uvula visible;
Class III—soft palate and base of uvula visible;
Class IV—soft palate completely not visible.

According to Mallampati's classification, classes III and IV are indicative of an airway that is more difficult to intubate.

The Cormack and Lehane classification, on the other hand, is based on the degree of visualization of the larynx with the laryngoscope, as follows:
Grade I—the glottis is clearly visible;
Grade II—only the posterior part of the glottis is visible;
Grade III—only the epiglottis can be seen, but no part of the glottis is visible;
Grade IV—the epiglottis and glottis cannot be visualized.

Thus, grade IV in Cormack's classification is also considered the most difficult for intubating the patient.

Considering the difficulty of intubating patients in general, and especially those with a difficult airway (DA), the present invention was created.

### STATE OF THE ART

The use of a laryngoscope during intubation can have some disadvantages, such as causing trauma to the incisor teeth, especially the upper incisors, due to the force often exerted by the flange of the Macintosh blade during intubation.

In addition, with the use of the laryngoscope, it is necessary to elevate the larynx and traction the chin. Manipulation of the larynx and chin with sufficient force to expose the vocal cords can damage tissue and nerves in the region, causing neurological and cardiac alterations as well as local bleeding and edema. Complications of laryngoscopy include hypertension (HTN), dysrhythmias, eye trauma, dental trauma, laryngospasm, bronchospasm, perforation of the airway or esophagus, bleeding, edema and airway obstruction (“Complications of Managing the Airway Jan-Henrik Schiff”, Carin A. Hagberg, in Benumof and Hagberg's Airway Management, 2013).

The present invention allows intubation in a very simple and practical way without the need to elevate the larynx and chin, reducing the mobilization of nerves and tissues, making the procedure easier for doctors and reducing the risk of complications compared to the use of a laryngoscope.

Thus, intubation with a laryngoscope requires a great deal of skill on the doctor's part and is often difficult even for the most experienced doctors. In order to solve this problem in the state of the art, the present invention was created. Through the present invention, it is possible to intubate the patient in a very simple and practical way, without the need to use a laryngoscope.

In order to avoid the use of a laryngoscope, especially in patients with a difficult airway (DA), there are semi-flexible (malleable) illuminated stylets available in the state of the art. These stylets act as a guide and have a light emitter in their distal portion. The light allows visualization of the soft tissues of the neck through the principle of transillumination, that is, the light passes through the patient's soft tissues, allowing the doctor to know where the tip of the stylet is. The purpose of this is to direct the tip of an endotracheal tube into the patient's trachea.

However, there is no camera for visualizing the patient's airways, especially the vocal cords. Therefore, the use of a lighted stylet is inadvisable when there are tumors, polyps or retropharyngeal abscesses, or any foreign body in the upper airway. In addition, due to the need to visualize transillumination, it is not recommended to use the lighted stylet in the presence of sunlight or in very bright environments.

Other state of the art stylets have a camera in their distal portion in order to visualize the patient's upper airways. However, they are flexible so that the patient needs to be laryngoscoped in order to perform intubation. In addition, these stylets act as a guide for the endotracheal tube. In this way, the endotracheal tube covers the stylet camera during the passage of the endotracheal tube between the patient's vocal cords (one of the most important and difficult moments of intubation). Therefore, these state of the art stylets, in a very disadvantageous way, do not allow visualization through the camera of the passing tube through the patient's vocal cords.

This stylet does not require the use of a laryngoscope as it is rigid.

While not using the laryngoscope, this stylet allows perfect visualization of the passage of the endotracheal tube through the patient's vocal cords during intubation.

This advantage provided by the present invention is obtained by the fact that the guide wire can be detached from the stylet during the intubation process. More specifically, in the present device, the semi-open channel of the guide wire allows it to be detached from the stylet as the endotracheal tube is introduced. This feature of the present invention will be better understood during the detailed description of the device.

### INVENTION SUMMARY

The present invention then discloses a stylet for endotracheal intubation fitted with a camera at the distal end comprising a rigid elongated element in a general “L” shape, having a substantially longitudinal axis through which a proximal handling portion is configured.

A distal portion forms an angle of 50 to 90° with the posterior portion, forming a knee that joins the posterior and distal portions. The stylet also has a semi-open channel along its entire length, which empties between the distal and proximal ends. This semi-open channel allows a guide wire to be attached and detached.

A distal portion forms an angle of 50 to 90° with the posterior portion, forming a knee that joins the posterior and distal portions. The stylet also has a semi-open channel along its entire length, which leaks at the distal and proximal ends. This semi-open channel allows a guide wire to be attached and detached.

### BRIEF DESCRIPTION OF THE DESIGNS

The figures below show an exemplary realization of the present invention.

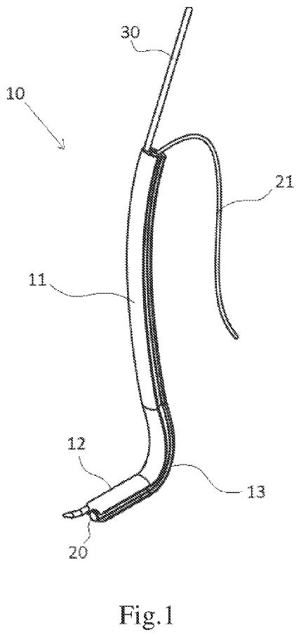

FIG. 1 shows a perspective view of a realization of the present invention.

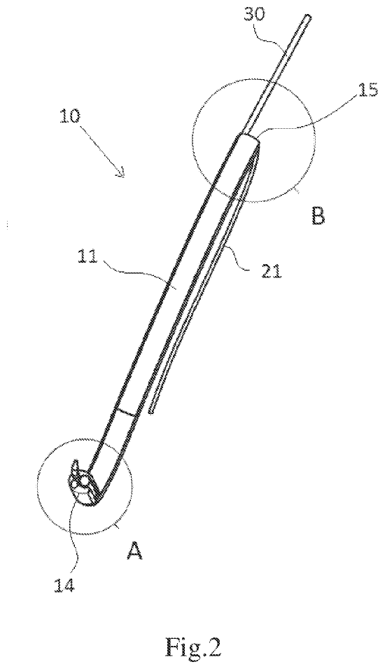

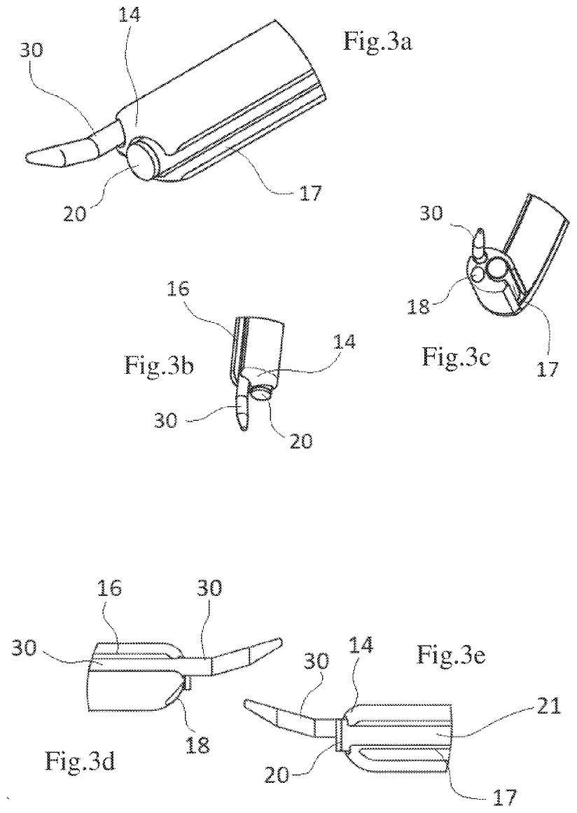

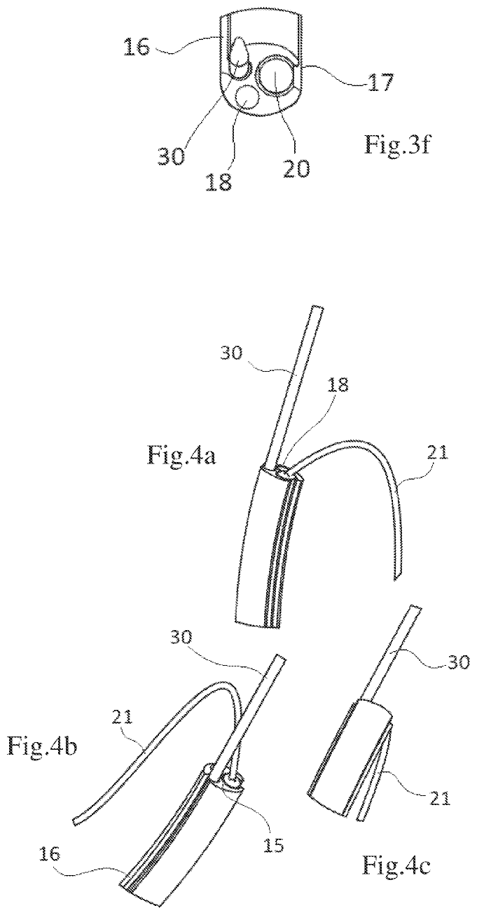

FIG. 2 shows another perspective view with indications of the distal (A) and proximal (B) portions, which are detailed in FIGS. 3 and 4 respectively.

FIG. 3 show the detail of the distal portion. FIG. 3a shows the detail of the camera and camera channel. FIG. 3b shows the guide wire and the semi-open channel for fitting the guide wire. FIG. 3c shows a perspective view of the three channels. FIGS. 3d and 3a show the channel details of the guide wire and camera respectively. FIG. 3f shows the front view of the distal end with details of the channels of the present invention.

FIG. 4 show the detail of the proximal portion. FIG. 4a shows the detail of the camera channel and FIG. 4b the guide wire channel. FIG. 4c shows the exit of the guide wire and camera cable.

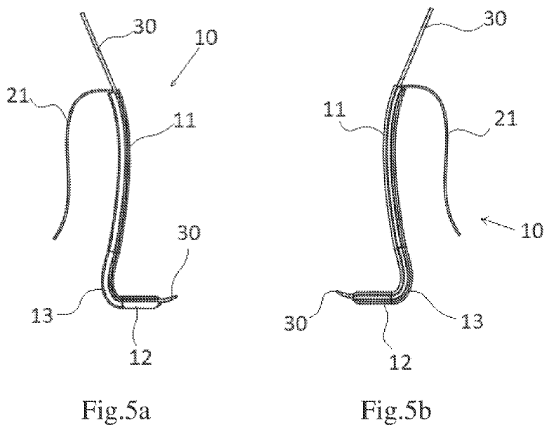

FIGS. 5a and 5b show the left and right side views respectively.

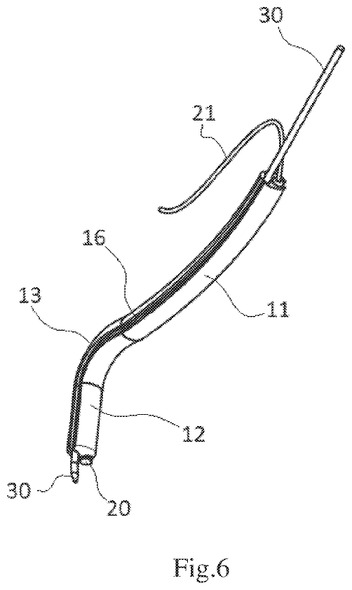

FIG. 6 shows another perspective view of a realization of the present invention.

### DETAILED DESCRIPTION OF THE INVENTION

The present invention teaches a stylet (10) necessary for endotracheal intubation, especially in patients with a difficult airway (DA). Although it is extremely useful in DA patients, the present invention is not limited to them and can be used to advantage in any patient. The invention makes the intubation procedure much simpler and more practical. It also reduces the chances of trauma to the patient, especially to the upper incisor teeth and the mucous membrane of the mouth and oropharynx.

When introducing the stylet (10) into the patient's oral cavity, the gentle curve of the knee (13) causes the end (14) of the distal portion (12) equipped with a camera (20) to point directly at the patient's larynx, immediately visualizing the vocal cords.

One of the greatest difficulties in endotracheal intubation is precisely the passage of the endotracheal tube (not shown) through the patient's vocal cords. Considering the general “L” shape of the stylet of the present invention, the camera (20) visualizes exactly this anatomical region, helping the professional in the intubation procedure.

The stylet (10) is inserted or fitted into a guide wire (30) through a semi-open channel (16). The guide wire (30) is then inserted into the opening or slot in the channel (16), which can be done in two ways.

The first way is done by inserting the guide wire (30) into the canal (16) from the proximal end (15). In this case, the professional holds the stylet (10) with one hand and introduces the guide wire (30) with the other hand into the semi-open canal (16) in the longitudinal direction of the proximal portion (11) of the stylet (10). The professional then pushes the guide wire (30), which slides inside the semi-open channel (16) until it comes out at the distal end (14).

The second way of inserting the guide wire (30) into the stylet (10) is by mechanically pressing it into the slot in the semi-open channel (16).

Thus, the slot in the semi-open channel (16) serves to engage and disengage the guide wire (30), acting as a pressure lock.

In both forms of fitting, the walls of the semi-open channel (16) exert a small mechanical pressure on the guide wire (30) sufficient to spontaneously keep it inside the semi-open channel (16). Despite this small pressure, the semi-open channel (16) allows the guide wire (30) to slide inside the stylet (10).

The stylet (10) must be fitted with the guide wire (30) for the procedure to begin. Thus, intubation begins by positioning the stylet (10) in the patient's oral cavity with the proximal end (15) pointed towards the patient's vocal cords, with the camera (20) capable of capturing images of the patient's laryngeal anatomy. The images are taken by a cable (21) to project the image onto a video monitor (not shown) which will assist the professional throughout the intubation process.

After the stylet (10) has been positioned on the patient, the professional pushes the guide wire (30) in a longitudinal direction from the proximal portion (11) in order to pass it through the patient's vocal cords. The passage of the guide wire (30) through the vocal cords is aided, in a very comfortable and practical way, by the images produced by the camera (20).

After passing the guide wire (30) through the patient's vocal cords, the professional inserts the endotracheal tube through the guide wire (30). In this way, the guide wire (30) remains inside the endotracheal tube, and the professional slides the endotracheal tube along the guide wire (30) in its longitudinal direction. In other words, the professional runs the endotracheal tube in the direction of the stylet (10).

When the endotracheal tube reaches the proximal end (15) of the stylet (11), the professional laterally detaches the guide wire (30) from the semi-open channel (16) to position the endotracheal tube wall between the stylet (10) and the guide wire (30).

In other words, by detaching the guide wire (30) from the semi-open channel (16), a space is created between the channel (16) and the guide wire (30). The professional then pushes the endotracheal tube towards the patient, positioning the endotracheal tube between the channel (16) and the guide wire (30).

As the guide wire (30) has only been partially detached from the semi-open channel (16) there is a place where it is still attached to the stylet (10). The healthcare professional, by continuing to push the endotracheal tube towards the patient, will cause its edge to meet the place where the guide wire (30) is still attached to the semi-open channel (16). The edge of the endotracheal tube is then positioned in the bifurcation created between the semi-open channel (16) and the guide wire (30). As the professional continues to push the endotracheal tube towards the patient, the guide wire (30) gradually detaches from the semi-open channel (16).

In summary, as the professional pushes the endotracheal tube towards the patient, the force exerted by the edge of the endotracheal tube at the bifurcation (that is, at the fitting limit) between the guide wire (30) and the semi-open channel (16) causes the guide wire (30) to detach from the stylet (10).

The professional continues pushing the endotracheal tube and detaching the guide wire (30) from the proximal portion (11) into the patient's oral cavity. Then they continue pushing the endotracheal tube and detaching the guide wire (30) from the knee (13) and the distal portion (12) until they reach the distal end (14), when the guide wire (30) is completely detached from the stylet (10).

At this point, the guide wire (30) has already been passed through the patient's vocal cords, and the professional continues to push the endotracheal tube through the vocal cords. This passage is aided by the images produced by the camera (20).

This is a great advantage of the invention, since the guide wire (30) is completely detached from the stylet (10), it is possible to use the images from the camera (20) to help the endotracheal tube pass through the patient's vocal cords, since the camera (20) is not covered by the endotracheal tube.

Once the endotracheal tube has been passed through the vocal cords, the professional can remove the stylet (10) and the guide wire (30), and the procedure is completely finished.

In the state of the art, this procedure may require the help of a professional to be conducted. With the use of the stylet of the present invention, intubation can be conducted by a single professional in a simple and quick manner.

The present invention is also equipped with a working channel (18) which has the function of sucking in saliva, mucus, blood, etc. by vacuum (in the direction of the patient's external environment). In the opposite flow direction, the working channel (18) can be used to introduce saline to clean the larynx or to introduce anesthetic to the patient's vocal cords.

Thus, the working channel (18) has the exemplary function of cleaning, aspirating and introducing anesthetics.

In other words, saline can be introduced through the working channel (18) and then the saline suctioned out to clean the larynx. Otherwise, the working channel (18) can only be used to extract excess mucus or saliva from the patient.

Another function of the working channel (18) is the introduction of anesthetics. In an intubation, an anesthetic is usually introduced (for example, topical Xylocaine or spray). However, this anesthetic may not reach the patient's vocal cords. Through the working channel (18), it is possible to advantageously introduce anesthetics into the patient's vocal cords during intubation, all with the same stylet (10).

Alternatively, the channel (17) through which the camera cable (21) passes can also be semi-open. A semi-open channel (17) for the camera (20) has the advantage of being able to detach the cable (21) and camera (20) for sterilization of the stylet (10) after the endotracheal intubation procedure.

Alternatively, the proximal portion (11) can have one or more curvatures in order to better adapt to the patient's anatomy. Likewise, the knee (13) can have a more or less gentle curve according to the patient's anatomy.

The measurements of the proximal (11), distal (12) and knee (13) portions, as well as the angulation between the proximal (11) and distal (12) portions, can be adjusted to suit different types of patients. These measurements can take into account the patient's weight and height, for example.

Thus, the present invention can be used on children, newborns, adolescents and adults, adjusting its dimensions to suit their respective anatomies.

In order to better identify the elements of the present invention, the respective numerical references follow:
10—stylet;
11—proximal portion;
12—distal portion;
13—knee;
14—distal end;
15—proximal end;
16—semi-open channel for attaching and detaching the guide wire;
17—camera channel;
18—working channel;
20—camera;
21—camera cable;
30—guide wire.

1. A stylet (10) for endotracheal intubation equipped with a camera (20) at the distal end (14) characterized by comprising a rigid elongated element in a general “L” shape, having a substantially longitudinal axis through which a proximal portion (11) is configured for handling; a distal portion (12) forming an angle of 50 to 90° with the proximal portion (11), forming a knee (13) which joins the proximal portion (11) and the distal portion (12); the aforementioned stylet (10) is also equipped throughout its length with a semi-open channel (16) which leaks at the distal end (14) and the proximal end (15), with the aforementioned semi-open channel (16) allowing a guide wire (30) to be attached and detached.

2. A stylet (10) for endotracheal intubation, according to claim 1, characterized by being further provided with an internal and continuous bidirectional working channel (18) along the entire length of the stylet with one opening in the distal portion (12) and the other in the proximal portion (11).
